(19) United States
(12) Patent Application Publication
(10) Pub. No.: US 2011/0195112 A1
(43) Pub. Date: Aug. 11, 2011

(54) USE OF PLANT-DERIVED RECOMBINANT GROWTH FACTORS IN SKIN CARE

(76) Inventors: Bjorn Larus Orvar, Kopavogur (IS); Einar Mantyla, Reykjavik (IS)

(21) Appl. No.: 13/001,642

(22) PCT Filed: Jun. 30, 2009
(86) PCT No.: PCT/IS2009/000003

§ 371 Date: Apr. 4, 2011

(30) Foreign Application Priority Data
Jun. 30, 2008 (IS) 8741
Oct. 9, 2008 (IS) 8764

(51) Int. Cl. A61K 38/18; A61K 9/127; A61K 38/20; A61K 38/19; C07K 14/54; A61P 17/00; A61K 8/64; A61Q 19/00; A61Q 19/08

(52) U.S. Cl. 424/450; 514/7.6; 514/9.6; 514/8.1; 514/8.2; 514/7.7; 514/9.1; 514/8.8; 424/85.2; 424/85.1; 514/9.2; 530/351

(57) ABSTRACT

Cosmetic and therapeutic compositions for skin care, containing a transgenic plant extract containing a growth factor, or a growth factor purified from transgenic plants, or a mixture of growth factors derived from transgenic plants as extracts or in purified form, for use in topical therapeutic and/or cosmetic applications. Importantly, this invention makes safer growth factors available for use for cosmetic and topical treatment. These growth factors do not carry the risk of unwanted contaminants and transmissible agents that can result from animals or animal cell based expression systems, and the recombinant growth factors that plant expression systems provide are post-translationally modified proteins.

### FIELD OF THE INVENTION

The present invention generally relates to cosmetic and pharmaceutical compositions comprising growth factors for skin care and methods for making cosmetic products. In particular, this invention relates to recombinant non-plant growth factors, preferably human or mammalian growth factors, obtained from transgenic plants and their use in cosmetic and pharmaceutical products.

### BACKGROUND OF THE INVENTION

Skin is the biggest organ of the human body carrying out various functions such as protection, barrier, temperature controlling, excretion and respiration. It performs various functions such as protection, barrier, temperature controlling, excretion and respiration. With time and ageing, those functions rapidly decline, and a variety of physiological changes occur to the skin. These changes are manifested in the decrease in the thickness of epidermis, dermis and subcutaneous tissue, which are the main components of skin. Changes in lipid composition undermine the moisture barrier role of lipid layers and resulting in the dryness of skin. Further, with age, the occurrence of age spots, freckles, pigmentation or various skin lesions also increases. Environmental components such as pollution and UV-rays, can speed up the ageing of the skin. Reactive oxygen species and free radicals and some physiological states such as fatigue or stress are particularly detrimental to proteins, nucleic acids and membrane lipids, leading to the aging of the skin. Accordingly, there have been many studies on the occurrence of the wrinkles, age spots or freckles, the loss of skin elasticity, the pigmentation, and the dryness of skin.

A variety of cosmetic compositions have been developed in order to prevent or slow down the problems of aging of the skin and skin wrinkles with the aim of improving wrinkles, sagging and the reduction in elasticity of skin caused by sunlight. Japanese Patent Laid-open Publication No. Hei 5-246838 discloses a method for improving wrinkles of skin by the synthesis of collagen. It teaches that the activity of collagenase that decomposes collagen to promote collagen metabolism might be reduced with aging, leading to the increase of cross-link collagens and the increase of skin wrinkles.

Growth factors are key players in regulating proliferation and differentiation of cells and are involved in restructuring the epidermis and basal lamina upon injury or damage. They are important for the renewal of cells and thus, can counteract several aspects of aging.

Growth factors are key players in maintenance of tissue integrity and in cell to cell communication, thus playing a protective role in fighting degeneration of epidermal tissue.

Fibroblast growth factors have proliferative effects on epithelial cells and have been observed to accelerate bone and wound healing in animal models.

Fibroblast growth factor 4 plays a central role during embryonic limb development; in vitro, FGF-4 is mitogenic for fibroblasts and endothelial cells and it has been shown to be a potent angiogenesis promoter in vivo.

Fibroblast growth factor 5 plays a major role during prenatal development and in postnatal growth and regeneration of various tissues, promoting cellular proliferation and differentiation; notably plays a role in the regulation of the hair growth cycle.

Fibroblast growth factor 6 plays a central role in growth and regeneration of a variety of tissues, by promoting cellular proliferation and differentiation; a potent mitogen for fibroblasts, it is important in skeletal muscle regeneration and may have angiogenic activity.

Fibroblast growth factor 8 plays a central role in growth and regeneration of a variety of tissues, by promoting cellular proliferation and differentiation and mediates epithelial-mesenchymal transitions.

Fibroblast growth factor 9 plays a major role during embryonic development and postnatal growth and regeneration of various tissues, promoting cellular proliferation and differentiation.

Heparin-binding EGF-like growth factor signals through the EGF receptor and stimulates the proliferation of smooth muscle cells, fibroblasts, epithelial cells and keratinocytes; produced in monocytes and macrophages. It may play an important role in wound healing.

Interleukin 4 is an anti inflammatory and immunosuppressive cytokine and shows a protective effect towards extracellular matrix degradation. Combination of IL-4 and IL-10 used for treatment of mice with arthritis appeared to markedly protect cartilage destruction.

Interleukin-15 appears to function as a specific maturation factor for NK-cells; stimulates proliferation of the established T-cell line CTLL-2 and CD8(+) memory T-cells require IL15 for proliferation.

Noggin is hypothetized to play an important role in the initiation of new hair growth wave in postnatal skin and in apoptosis-driven hair follicle regression in normal skin; exogenous introduction of noggin can restore hair follicle development in lama5(−/−) skin.

Placenta growth factor is a potent angiogenic factors stimulating angiogenesis without significant enhancement of vascular leakage and inflammation; it is expressed during cutaneous wound healing and Improves wound closure by enhancing angiogenesis. Expression of SCF in humans and animals is correlated with the ability of dermal papilla cells inducing hair follicle regeneration. Hair pigmentation is regulated by several factors including the interaction of SCF with its class III receptor tyrosine kinase, c-kit.

Flt3 ligand is a ligand for the FLT3 tyrosine kinase receptor and belongs to a small group of growth factors that regulate proliferation of early hematopoletic cells. Multiple isoforms of Flt3 ligand have been identified. Flt3 ligand binds to cells expressing the tyrosine kinase receptor Flt3. Flt3 ligand alone cannot stimulate proliferation, but synergizes well with other CSFs and interleukins to induce growth and differentiation and is therefore suitable addition to compositions containing one or more growth factors.

Growth factors can promote cellular renewal and proliferation and are a natural component of the healing process of wounds.

U.S. Pat. No. 5,618,544 incorporated herein by reference in its entirety, discloses a cosmetic composition comprising EGF, TGF-a and FGF for decreasing cutaneous senescence and improving the appearance of skin.

U.S. Pat. No. 6,589,540 teaches that EGF remarkably enhances the effect of retinol used in and cosmetics, and also effectively alleviates the skin irritation of retinol.

It is recognized that growth factors can have beneficial effects on various skin disorders and skin injuries and counteract effects of aging that are the result of impaired or deteriorating protective mechanisms at cellular level.

Growth factors are released at wound site during coagulation phase, and act as chemo-attractants for neutrophils, macrophages and fibroblasts. These cells play an important role in killing bacteria and removal of necrotic debris at the wound site. Activated macrophages release in turn growth factors that promote angiogenesis and communicate with the B-cell and T-cell mediated immune responses. Macrophages secrete growth factors that stimulate fibroblasts to produce new extracellular matrix, and stimulate angiogenesis. Epithelization proceeds as keratinocytes divide and cover the wound bed. Epidermal growth factor stimulates the proliferation of fibroblasts and keratinocytes. Thus, it is well established that growth factors are important mediators of healing process and studies indicate that some growth factors may be beneficial for treating infected diabetic ulcerations.

Growth factors isolated from animal tissue or blood carry the risk of unwanted contaminants and transmissible agents, such as but not limited to viruses, virions, prions, other co-purifying growth factors. The same risk of contaminating transmissible agents and endogenous growth factors is present in growth factors produced in animal or human cells by biotechnological means. Growth factors produced with biotechnological means in bacteria pose the risk of carry-over of bacterial endotoxins that are known to be pyrogenic and disturb the immune system. Furthermore, bacteria are unable to glycosylate proteins, which in several cases is known to make them less stable and more prone to degradation by proteases. The risk of transmissible agents, endotoxins or contaminants is clearly of concern for the use of growth factors produced in bacteria, yeast- or animal cells to treat open wounds.

There is continued demand for growth factors and other biologically active proteins of high quality, prepared such as to minimize or eliminate the above problems and disadvantages.

### SUMMARY OF THE INVENTION

Recombinant growth factors and cytokines produced in plants are free from transmissible infectious agents such as animal or human viruses, virions and prions and bacterial endotoxins. There are no reported cases of plant diseases that could cause diseases in man, in contrary to numerous animal diseases that can infect man. Thus, plants constitute a much safer production organism than the above mentioned cell types for the production of growth factors. Plants lack an immune system comparable to that of animals that requires the action and participation of growth factors as signaling elements. Plants do not produce themselves growth factors similar to animal or human growth factors. Plants are able to glycosylate proteins, which improves the stability of those proteins and can affect their activity, and therefore plant systems are able to produce superior growth factors compared to those produced in bacteria. Production of growth factors in plants with biotechnological means according to the invention circumvents these safety and purity problems. Plant-derived growth factors whether in extract or in purified form are therefore safer and cleaner for use in cosmetics or topical therapeutics than growth factors produced with current production methods.

Plants produce a number of proteins that play a protective role in the plant and alleviate stress caused by abiotic and biotic factors, such as dehydration and oxidative stress. Several of these proteins accumulate specifically in the seeds of a plant upon seed maturation that involves dehydration of the cellular tissue. Dehydrins are a class of proteins that accumulate in response to stress such as drought or as a part of a maturing process such as seed development. Consequently, by providing growth factors for cosmetic products through expression in transgenic plants and preparing a plant extract from said plants, containing said growth factor, the extract provides not only the beneficial growth factor through a bio-risk free process but also provides the growth factor in a matrix that can provide as a bonus effect other beneficial components derived from the plant in the same plant extract, originating from the same transgenic plant.

It is an object of the present invention to provide compositions for skin care, which can have either or both cosmetic or therapeutic use, comprising a transgenic plant extract comprising a recombinant growth factor, or a recombinant growth factor purified from transgenic plants, or a mixture of growth factors derived from transgenic plants as extracts or in purified form, for use in topical therapeutics and cosmetics. Importantly this invention makes safer growth factors available for use for cosmetic and topical treatment.

These plant produced growth factors may be glycosylated in plants when they carry glycosylation sites in their amino acid backbone, a feature that is known to improve stability of proteins and may affect their biological activity. As mentioned above, it is a drawback of other expression systems, such as prokaryotic systems, that those systems lack a mechanism for glycosylation of expressed heterologous proteins. Not only are plants capable of glycosylating heterologous proteins, but the glycosylation mechanisms differ from that found in animals such as humans. While this may in some clinical applications be a disadvantage, it is not clinical relevance for many other applications and may in fact contribute to the quite surprising stability of the plant derived heterologous proteins of the invention.

It has been a critical issue for practical use of sensitive biomaterials in cosmetic products and in particular growth factors, that these materials are unstable. Consumers generally expect a reasonably long shelf life of cosmetic products, and wish to store such products at room temperature, i.e. it is a significant commercial disadvantage if cosmetic products are to be stored refrigerated. Cosmetic producers have begun labeling their products with a shelf life label but generally give little or no information about preferred storage conditions. As shown in the accompanying examples, growth factors and growth factor extracts provided by the present invention show surprisingly good stability even at rather extreme conditions.

Production of active ingredients such as growth factors for compositions for cosmetic and/or therapeutic topical use is made more economical by the present invention. The plant expression systems allow scale-up production of desired recombinant proteins and simple and robust purification schemes can be used to extract the protein from the cellulosic plant material to provide a useful plant extract. In many cases the protein need not be extensively purified, as many of the bulk components in such plant extract are not harmful to the skin and may even be beneficial, as mentioned above. In certain useful embodiments the plant extract used in the compositions of the invention comprise in the range of about 0.01% to 70% of the growth factor of interest, measured as wt % of the total protein content of the extract, such as in the range of about 0.1 to about 30% of total protein, including the sub-ranges 0.1-1% and 0.1-5%, and 1-10%, 1-30% of total protein, and suitable intermediate values, such as but not limited to about 0.1% or about 1% of total protein in the extract. In other embodiments, the extract may more substantially purified and contain upto about 90% of the growth factor protein or more, such as about 95% or more, or 99% or more, of total protein content in the extract. Thus, the extract according to the invention may in certain embodiments generally comprise in the range of about 0.01% to about 99.9% of said growth factor, and preferably in the range of about 0.1 to about 99.9%, including sub-ranges, such as e.g. from about 0.01 to about 70% and about 0.1 to about 40% and the range of about 40 to about 70% of growth factor, as % of total protein.

More specifically, it is an object of the present invention to provide a cosmetic and/or therapeutic skin-care composition comprising a recombinant growth factor, and more preferably any of the herein listed growth factors and optionally in a composition with other, naturally occurring, plant-based beneficial polypeptides, such as dehydrins and globulins in the extract. These seed proteins have a protective function at the cellular and biochemical level in plants and in the unique combination with a growth factor, as an object of this invention, they provide nurturing and healing conditions and alleviate dehydration and oxidative stress at a cellular level.

It is further an object of the present invention to provide a skin care composition suitable for the cosmetic or clinical treatment of acne, the improvement of skin wrinkles, age spots, freckles, blotches or other pigmentation, and the moisturizing of skin and wound healing.

In another aspect, the present invention provides a method of manufacturing a topical cosmetic and/or therapeutic product comprising providing a transgenic plant extract comprising a recombinant heterologous growth factor. The plant-produced non-plant originating recombinant growth factor may be selected from the group consisting of Epidermal Growth Factor (EGF), Vascular Epithelial Growth Factor (VEGF), Platelet-Derived Growth Factor (PDGF), Erythropoietin (Epo), Fibroblast growth factors 4, 5, 6, 8 and 9 (FGF4, FGF5, FGF6, FGF8 and FGF9), Fibroblast Growth Factors a and b, Flt3 ligand, Heparin binding-EGF (Hb-EGF), Insulin-Like Growth Factor-I (IGF-I), Insulin-Like Growth Factor-II (IGF-II), Interleukin-1 (IL-1; including IL-1 alpha and IL 1-beta), Interleukin-2 (IL-2), Interleukin-4 (IL-4), Interleukin-7 (IL-7), Interleukin-6 (IL-6), Interleukin-8 (IL-8), Interleukin-10 (IL-10), Interleukin-15 (IL-15), Interleukin-18 (IL-18), Interleukin-20 (IL-20), leukemia inhibitory factor (LIF), Noggin, Placenta growth factor-1 (PlGF-1), Stem cell factor (SCF), Transforming growth factor alfa and beta (TGF a and TGF b), including TGF b3 Tumor Necrosis Factor-a (TNF-a), Tumor Necrosis Factor-b (TNF-b), Interferon-g (INF-g), Granulocyte Colony Stimulating Factor (G-CSFs), Granulocyte Macrophage Colony Stimulating Factor (GM-CSF), Macrophage Colony stimulating factor (M-CSF),

Nerve Growth Factor (NGF), Keratinocyte Growth Factor (KGF), Bone morphogenesis Protein (BMP-4), and Thymosin beta 4. In certain preferred embodiments, the transgenic plant extract is a barley seed extract. The produced growth factors are in particular useful for making a cosmetic composition.

In yet a further aspect, the present invention provides one or more recombinant heterologous growth factor isolated from transgenic plants. The growth factors may also be used in other applications known to a skilled person in the art.

In a further aspect of the present invention, novel plant extracts containing growth factors are provided to be used for cosmetic and/or therapeutic purposes and as an active ingredient such as in healing ointments or other forms of topical pharmaceutical compositions.

### BRIEF DESCRIPTION OF FIGURES

FIG. 1 shows a stained gel and an immunoblot with transgenic plant extract containing mSCF and isolated mSCF from transgenic plant extract (see Example 2).

FIG. 2 shows a Western blot of recombinant interferon gamma produced in plant and in bacteria, treated with primary antibody against fucose (see Example 3).

FIG. 3 shows a Western blot of recombinant interferon gamma produced in plant and in bacteria, treated with primary antibody against xylose (Example 3).

FIG. 4 shows a stained gel with plant-made IL-1a (freeze-dried and reconstituted), which has been incubated at various temperatures and stored for varying times (see Example 4).

### DETAILED DESCRIPTION OF THE INVENTION

As used herein, a “plant-derived” growth factor or “growth factor derived from plant” indicates a recombinant growth factor obtained from a transgenic plant or progenies of a transgenic plant. The growth factor according to the present invention is generally a heterologous non-plant originating growth factor and may preferably be any human or non-human growth factor, such as preferably a mammalian growth factor, the gene of which has been introduced into said transgenic plant or progenitors of the plant, preferably using recombinant technology. The isolated growth factor may be used as an active ingredient in a cosmetic composition or a therapeutic topical composition.

Methods for introducing and expressing foreign genes in plants are well known in the art. A plant that can be genetically transformed is a plant into which heterologous DNA sequence, including DNA sequence for a coding region, can be introduced, expressed, stably maintained, and transmitted to subsequent generations of progeny. Genetic manipulation and transformation methods have been used to produce barley plants that are using herbicide resistance including, for instance, bialaphos or baste, or antibiotic resistance, such as hygromycin resistance, as a selectable marker.

Suitable cultivars are selected and a suitable method for introduction of foreign gene selected. The term “transformation” or “genetic transformation” refers to the transfer of a nucleic acid molecule into the genome of a host organism, resulting in genetically stable inheritance. Host organisms containing the transformed nucleic acid fragments are referred to as “transgenic” organisms. A “transgenic plant host cell” of the invention contains at least one foreign, preferably two foreign nucleic acid molecule(s) stably integrated in the genome. Examples of methods of plant transformation include Agrobacterium-mediated transformation (De Blaere et al. 1987) and particle-bombardment or “gene gun” transformation technology (Klein et al. (1987); U.S. Pat. No. 4,945,050).

WO 2006/016381 describes a particular useful Barley cultivar amenable for transformation and describes in detail suitable transformation methods. This document is incorporated herein in its entirety by reference.

WO 2005/021762 discloses methods for producing modified proteins in plant expression systems by making chimeric proteins that are readily purified on a large scale. This document is also incorporated herein in its entirety by reference.

Growth factors that are suitably produced and used according to the present invention may be selected from any of the above mentioned growth factors and more preferably from the group consisting of Fibroblast growth factors 4, 5, 6, 8 and 9 (FGF4, FGF5, FGF6, FGF8 and FGF9), Flt3 ligand, Heparin binding-EGF (Hb-EGF), interleukin 4 and 15 (IL-4, IL-15), leukemia inhibitory factor (LIF), Noggin, Placenta growth factor-1 (PlGF-1), Stem cell factor (SCF), Transforming growth factor beta 3 (TGF b3).

In certain embodiments of the invention, the polypeptide of interest being produced in the transgenic plant contains an affinity tag at either N-terminal or C-terminal of the polypeptide, or at both ends. Such a tag may include repetitive HQ sequence, poly-GST (Glutathione S-transferase), CBM (carbohydrate binding module) or any other useful affinity tag that simplifies purification of the heterologous peptide, allowing for affinity purification.

As mentioned above, the glycosylation mechanisms in plants differ from those found in animals such as mammals and is also different from the glycosylation systems in yeast, which is another common expression system for heterologous recombinant protein products. Plants are able to produce proteins with complex N-linked glycans. Plant glycoproteins have complex N-linked glycans containing α-1,3 linked core fucose and β-1,2 linked xylose residues not found in mammals. Plant glycoproteins lack the characteristic galactose (NeuAcα2, 6Galβ-1,4) containing complex N-glycans found in mammals, as plants do not contain β(1,4)-galactosyltransferases nor α(2,6)sialyltransferases. Also α-1,6 linked core fucose is never found.

Accordingly, the invention encompasses in an embodiment a cosmetic and/or therapeutic composition as described above comprising a plant extract comprising a recombinant non-plant originating growth factor, glycosylated with one more plant-specific glycans, including glycans comprising α-1,3 linked fucose and/or β-1,2 linked xylose.

Dosage: Suitable dosage of the cosmetically or therapeutically active ingredients in accordance with the present invention, for topical cosmetic and/or therapeutic application, the amounts of heterologous growth factor protein typically fall within the range from 0.01 to 100 ppm (μg/gram) of composition. Local cosmetic compositions for the treatment of skin ageing or loss of hair preferably comprise from 0.1 to 5 ppm of active substance in composition.

The length of treatment varies depending on the pathology or on the desired effect. In the case of scleroderma treatment the application ranges from 1 day to 12 months according to the pathology severity. In the case of a treatment against natural or early ageing of the skin, the application may range from 1 to 400 days, preferably for at least 30 days. Likewise, in the case of a treatment for preventing loss of hair or for promoting hair re-growth the application preferably ranges from 1 to 400 days.

Preferably the transgenic plant extract is prepared from grains of barley containing any one of the proteins on the said list, their mimetics or at least domains thereof that enable binding to, and activation of a growth factor receptor. The below included illustrating examples show transgenic barley extracts containing different growth factors, including Hb-EGF, mSCF, FGF5, IL4, and FGF6.

Numerous vehicles for topical application of cosmetic and pharmaceutical compositions are known in the art. See, e.g., Remington's Pharmaceutical Sciences, Gennaro, A R, ed., 20th edition, 2000: Williams and Wilkins Pa., USA. All compositions usually employed for topically administering cosmetic compositions may be used, e.g., creams, lotions, gels, dressings, shampoos, tinctures, pastes, ointments, salves, powders, liquid or semi-liquid formulation, patches, liposomal preparations, solutions, suspensions, liposome suspensions, W/O or O/W emulsions, pomades and pastes and the like. Application of said compositions may, if appropriate, be by aerosol e.g. with a propellant such as nitrogen carbon dioxide, a freon, or without a propellant such as a pump spray, drops, lotions, or a semisolid such as a thickened composition which can be applied by a swab. In particular compositions, semisolid compositions such as salves, creams, lotions, pastes, gels, ointments and the like will conveniently be used.

The compositions of the invention can be provided for parenteral, systemic or local use, comprising solutions, suspensions, liposome suspensions, W/O (water/oil) or O/W (oil/water) emulsions. In a preferred embodiment the active substance is formulated in a lyophilized form, mixed to suitable lyophilisation additives and ready to be redissolved with therapeutically acceptable diluents. Useful lyophilisation additives are: buffers, polysaccharides, sucrose, trehalose, mannitol, inositol, polypeptides, amino acids and any other additive compatible with the active substance. Diluents suitable for parenteral use are: water, physiological solutions, sugar solutions, hydroalcoholic solutions, oily diluents, polyols, like glycerol, ethylene or polypropylene glycol, or any other diluent compatible with the administration method as for sterility, pH, ionic strength and viscosity.

In the case of emulsions or suspensions, the composition may contain suitable surfactants of non-ionic, zwitterionic, anionic or cathionic type commonly used in the formulation of medicaments. Oil/water (O/W) hydrophilic emulsions are preferable for parenteral systemic use, whereas water/oil (W/O) lipophilic emulsions are preferable for local or topic use.

Moreover, the compositions of the invention may contain optional additives like isotonic agents, such as sugars or polyalcohols, buffers, chelating agents, antioxidants, antibacterials.

Liquid forms according to the invention can comprise solutions or lotions. These may be aqueous, hydroalcoholic, like ethanol/water, or alcoholic and are obtained by solubilising the lyophilised substance.

Alternatively, active substance solutions, may be formulated in form of gel by addition of known gelling agents, like: starch, glycerin, polyethylene, pentylene glycol, polypropylene glycol, poly(meth)acrylate, isopropyl alcohol, and hydroxystearate.

Other types of compositions for topical use are emulsions or suspensions in form of pomades, pastes, creams. W/O emulsions are preferable, providing a faster absorption. Examples of lipophilic excipients are: liquid paraffin, anhydrous lanolin, white vaseline, cetyl alcohol, stearyl alcohol, vegetable oils, mineral oils. Agents increasing cutaneous permeability, thereby facilitating the absorption, may advantageously be used. Examples of such agents are physiologically acceptable additives like polyvinyl alcohol, polyethyleneglycol or dimethylsulfoxide (DMSO).

Other additives used in the topic compositions are isotonic agents, like sugars or polyalcohols, buffers, chelating agents, antioxidants, antibacterials, thickeners, dipersants.

It follows that the preparations may further contain conventional components usually employed in preparations described herein, including oils, fats, waxes, surfactants, humectants, thickening agents, antioxidants, viscosity stabilizers, chelating agents, buffers, preservatives, perfumes, dyestuffs, lower alkanols, and the like.

Delayed-release compositions for local or systemic use may be useful, and comprise polymers like polylactate, poly(meth)acrylate, polyvinylpyrrolidone, methylcellulose carboxymethylcellulose and other substances known in the art. Delayed-release compositions in form of subcutaneous implants based on, e.g. polylactate or other biodegradable polymers may be useful as well.

Though the active substance is preferably packaged in lyophilised and hence stable form, the pharmaceutical compositions advantageously comprise substances stabilising the plant derived heterologous growth factor in the active mono-, di- or multimeric forms. Such stabilisers inhibit the formation of intermolecular disulfide bonds, thereby preventing the polymerisation of the active substance. However, the amount of stabiliser should be carefully measured in order to concomitantly prevent the reduction of the active substance to an inactive form. Examples of such substances are: Cystein, Cysteamine, or glutathione in reduced form.

Non-limiting examples of oils include fats and oils such as olive oil and hydrogenated oils; waxes such as beeswax and lanolin; hydrocarbons such as liquid paraffin, ceresin, and squalene; fatty acids such as stearic acid and oleic acid; alcohols such as cetyl alcohol, 1,2, Hexanediol, stearyl alcohol, lanolin alcohol, aminomethyl propanol, and hexadecanol; and esters such as isopropyl myristate, isopropyl palmitate and butyl stearate. As examples of surfactants there may be cited anionic surfactants such as sodium stearite, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrochloride solutions and lecithin; and nonionic surfactants such as glycerin monostearate, sorbitan monostearate, sucrose fatty acid esters, propylene glycol, pentylene glycol, monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g. the materials sold under the trademark “Pluronic”), polyoxyethylene castor oil, and polyoxyethylene lanolin. Examples of humectants include glycerin, 1,3-butylene glycol, 1,2, Hexanediol, caprylyl glycol and propylene glycol; examples of lower alcohols include ethanol and isopropanol; examples of thickening agents include xanthan gum, hydroxypropyl cellulose, acrylates/C10-30 Alkyl Acrylate Crosspolymer, hydroxypropyl methyl cellulose, polyethylene glycol, pentylene glycol and sodium carboxymethyl cellulose; examples of antioxidants include butylated hydroxytoluene, butylated hydroxyanisole, propyl gallate, citric acid and ethoxyquin; examples of chelating agents include disodium dentate and ethanehydroxy diphosphate; examples of buffers include citric acid, sodium citrate, boric acid, borax, and disodium hydrogen phosphate; and examples of preservatives are methyl parahydroxybenzoate, tropolone, ethyl parahydroxybenzoate, dehydroacetic acid, salicylic acid and benzoic acid. These substances are merely exemplary, and those of skill in the art will recognize that other substances may be substituted with no loss of functionality.

The invention further provides methods for manufacturing compositions as described herein, the methods generally comprising providing a plant extract from a transgenic plant expressing a heterologous growth factor as described herein, preferably a growth factor as listed above and combining said extract with at least one cosmetically acceptable excipient, and preferably one or more of the above mentioned excipients. The method can further comprise the steps of harvesting the transgenic plant, separating the growth factor fom plant material and extracting a plant extract therefrom containing the growth factor. In the case of preferred plants that express the heterologous growth factor in its seeds, the separation can suitably include the steps of collecting the seeds, which can be conveniently stored for extended time without seriously affecting the activity of the growth factor.

### EXAMPLES

### Example 1

### Partially Purified Transgenic Plant Extract Containing Recombinant Growth Factor and Dehydrins

A transgenic plant extract was prepared by milling harvested transgenic barley seeds containing a recombinant growth factor murine Stem Cell Factor (mSCF), in a mill to obtain fine powder (flour). Extraction buffer added (50 mM potassium phosphate pH 7.0) to the milled barley flour in a volume/weight ratio of 5/1 of extraction buffer to milled flour. The resulting solution was stirred for 60 minutes at 4° C. Solids were separated from the liquid extract by centrifugal force, centrifuging at 8300 rpm in a refrigerated Centrifuge (Heraeus Primo R) or more, for 15 minutes, and the supernatant decanted off to a fresh vial. The growth factor content of the extract was analysed by SDS-PAGE and Western blotting with a specific antibody. In this experiment the mSCF content was about 0.1% of the protein content of the unpurified extract.

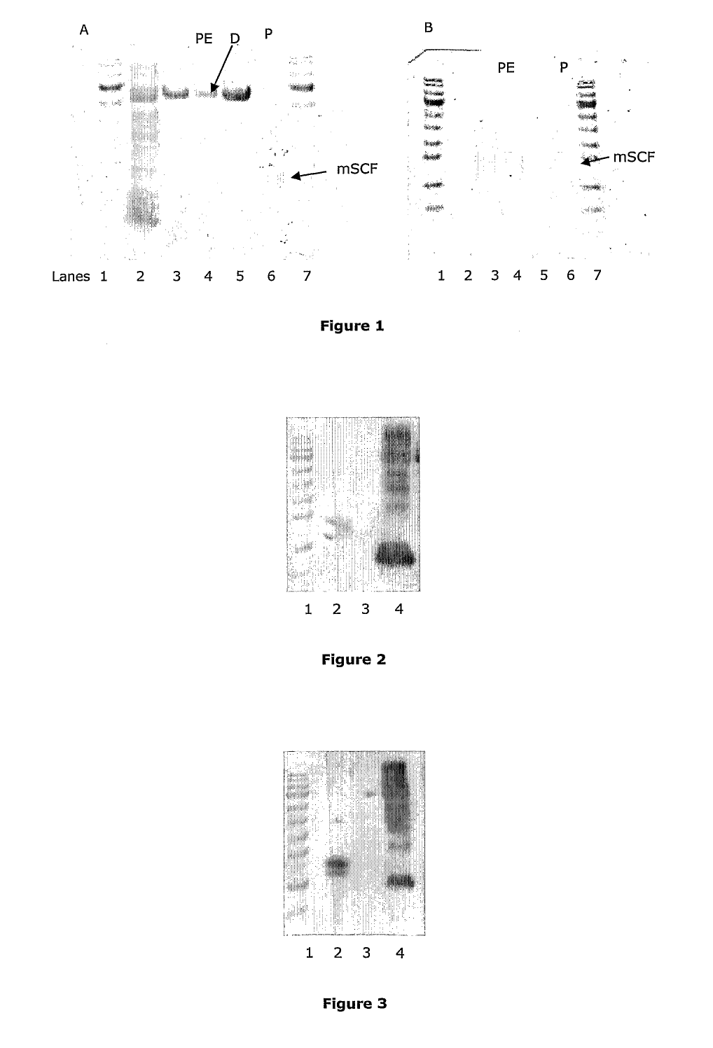

The resulting transgenic barley seed extract from Example 1 was processed further by adding to the extract an IMAC chromatography resin that effectively binds the mSCF. The mixture of extract and resin was stirred in 50 mM potassium phosphate, 0.5 M NaCl, 50 mM imidazole; pH7.0 at +4° C. for 60 minutes. The IMAC resin was separated from the liquid by centrifugation at 5000×g for 15 minutes. The liquid phase was decanted off and the resin was resuspended in washing buffer (50 mM potassium phosphate, 0.5 M NaCl, 50 mM imidazole; pH7.0) and spun down and the liquid phase decanted off the resin. The washing was repeated for 3 times. The resin was resuspended in elution buffer containing imidazole (50 mM potassium phosphate, 0.5 M NaCl, 500 mM imidazole; pH7.0) to elute the mSCF off the resin and after centrifugation the supernatant was decanted off the resin and run through gel filtration chromatography (desalting) for buffer exchange. The resulting protein peak was analysed on SDS-PAGE and Western blot. In this case the mSCF was present as approximately 40% of the protein extract. The partially purified Extract is shown in FIG. 1 lane marked PE.

### Example 2

### Purification of Recombinant Growth Factor mSCF Purified from Transgenic Barley Seed Extract

To further isolate a growth factor, in a purified form: The IMAC elute from Example 1 is, after buffer exchange with gel filtration, applied to an ion exchange column Sepharose FF and the proteins in the extract were separated by stepwise elution increasing the NaCl content of the elution buffer. It was possible in this manner to successfully separate the growth factor from the dehydrin. The band is fuzzy due to glycosylation of the mSCF. As shown in FIG. 1 a growth factor can be purified to a high purity, >95% (lane 6) in this manner resulting in an isolated and purified mSCF isolated and purified from a transgenic plant extract.

FIG. 1 shows transgenic plant extract and purification and isolation of murine Stem Cell Factor (mSCF) from transgenic plant extract.

A) Coomassie blue stained SDS-PAGE gel staining total proteins present in the extract and at different purification steps.
B) Western blot of mSCF containing extracts.

Lanes: 1 and 7 size markers; 2,3: IMAC elute; 4: desalted IMAC elute; 5: 50% NaCl elute from IEC; 6: 100% NaCl elute.

PE: partially purified Plant Extract; P: Purified Protein; D: dehydrin, mSCF: murine Stem Cell Factor.

### Example 3

### Glycosylation of Recombinant Growth Factors Expressed in Plants

Human interferon gamma was expressed in barley, the extraction of total proteins was performed under reducing conditions in the presence of 170 mM NaCl, 1% 2-Mercaptoethanol, 10 mM Tris-HCl pH 8.0 and 1% Polyvinyl pyrrolidine (MW 360.000). After milling the sample, 5 ml of the extraction buffer was added to the extraction vial. The extract was clarified by centrifugation at 4000 rpm for 10 min at 4° C., followed by a tenfold centrifugal concentration in Ultrafree-4 concentrators with molecular 5 kDa cut-off (UFV4BCC00-Millipore Corp. Bedford, Mass., USA). A 100 μl sample of the clarified, concentrated extract was added to 100 μl of 2× sample buffer and the mixture placed in a boiling water bath for 5 min. After cooling, 10 μl of the sample was loaded on 12% polyacrylamide gel separated with SDS-PAGE. The results of the comparative example are illustrated with a Western blot (see FIG. 2) of recombinant IFN gamma produced in plant and in bacteria, separated by SDS-Polyacrylamide gel electrophoresis and electroblotted onto PVDF membrane. The membrane is treated with primary antibody against plant specific 1-3 fucose. This shows that the IFN gamma (showing 2 bands of differential glycosylation) carries 1-3 fucose whereas the bacterially produced IFN gamma in the adjacent lane shows no signal.

The Western blot is made with anti fucose primary antibody, 5 sec exposure time. Samples loaded as follows: lane 1: MW ladder, lane 2: plant produced IFN gamma, lane 3: bacterial produced IFN gamma, lane 4 plant extract (positive control).

A second blot of the same samples was made using anti-xylose antibody. FIG. 3 shows the Western blot of recombinant interferon gamma produced in plant and in bacteria, separated by SDS-Polyacrylamide gel electrophoresis and electroblotted onto PVDF membrane. The membrane is treated with primary antibody against plant-specific sugar xylose.

This shows that the IFN gamma (showing 2 bands of differential glycosylation) carries xylose, whereas the bacterially produced IFN gamma in the adjacent lane shows no signal due to lack of glycosylation.

The blot, shown in FIG. 3, had 5 sec exposure time with -anti fucose primary antibody Samples loaded as follows: lane 1: MW ladder, lane 2: pl. IFN gamma, lane 3: bacterial IFN gamma, lane 4 plant extract (positive control).

### Example 4

### Stability of Recombinant Growth Factors Expressed in Plants

In this example a stability test was performed for purified, reconstituted freeze-dried plant-made interleukin 1a (IL-1a) (containing one N-glycosylation site) incubated at various temperatures; refrigerated at +4° C., +37° C. and room temperature (RT) for up to 3 weeks. The results depicted in FIG. 4 show excellent stability of the growth factor at 37° C., RT and at +4° C. for several weeks.

According to descriptions by manufacturers of bacterially (E. coli) manufactured IL-1a, the reconstituted non-glycosylated IL-1a, purified form from bacteria is only stable for one week at 2° C.-4° C. (Ref. http://www.cellsciences.com/PDF/CR1132.pdf) This example shows that plant-made growth factor, in this instance IL-1a, is substantially more stable than the same growth factor expressed in a bacterial system.

### Example 5

### Use of a Plant-Derived Growth Factor in a Compositions

The following examples illustrate formulations of the cosmetic composition according to the present invention but are not intended to limit the invention in any way.

Formulation 1: Skin softener (Skin lotion)

| Ingredients | Amounts (% by weight) |
|---|---|
| Hb-EGF (from transgenic plant) | 0.0001 |
| 1,3-Butyleneglycol | 6.0 |
| Glycerin | 4.0 |
| Oleyl alcohol | 0.1 |
| Polysorbate 20 | 0.5 |
| Ethanol | 15.0 |
| Benzophenone-9 | 0.05 |
| Flavor | 0.2 |
| Methylparaben | 0.2 |
| Imidazolidinyl Urea | 0.2 |
| Purified water | q.s. |

### Formulation 2: Nutrient Emulsion (Milk Lotion)

| Ingredients | Amounts (% by weight) |
|---|---|
| mSCF (from transgenic plant) | 0.0002 |
| Propyleneglycol | 6.0 |
| Glycerin | 4.0 |
| Triethanolamine | 1.2 |
| Tocopherylacetate | 3.0 |
| Liquid paraffin | 5.0 |
| Squalene | 3.0 |
| Makadamia nut oil | 2.0 |
| Polysorbate 60 | 1.5 |
| Sorbitan sesquioleate | 1.0 |
| Carboxyvinylpolymer | 1.0 |
| Flavor | 0.2 |
| Methylparaben | 0.2 |
| Imidazolidinyl Urea | 0.2 |
| Purified water | q.s. |

### Formulation 3: Nutrient Cream

| Ingredients | Amounts (% by weight) |
|---|---|
| FGF5 (from transgenic plant) | 0.0005 |
| Vaseline | 7.0 |
| Liquid paraffin | 10.0 |
| Wax | 2.0 |
| Polysorbate 60 | 2.0 |
| Sorbitan sesquioleate | 2.5 |
| Squalene | 3.0 |
| Propyleneglycol | 6.0 |
| Glycerin | 4.0 |
| Triethanolamine | 0.5 |
| Carboxyvinylpolymer | 0.5 |
| Tocopherylacetate | 0.1 |
| Flavor | 0.2 |
| Methylparaben | 0.2 |
| Imidazolidinyl Urea | 0.2 |
| Purified water | q.s. |

### Formulation 4: Massage Cream

| Ingredients | Amounts (% by weight) |
|---|---|
| IL4 (from transgenic plant) | 0.0002 |
| Propyleneglycol | 6.0 |
| Glycerin | 4.0 |
| Triethanolamine | 0.5 |
| Wax | 2.0 |
| Tocopherylacetate | 0.1 |
| Polysorbate 60 | 3.0 |
| Sorbitan sesquioleate | 2.5 |
| Cetearyl alcohol | 2.0 |
| Liquid paraffin | 30.0 |
| Carboxyvinylpolymer | 0.5 |
| Flavor | 0.2 |
| Methylparaben | 0.2 |
| Imidazolidinyl Urea | 0.2 |
| Purified water | q.s. |

### Formulation 5: Facial Pack

| Ingredients | Amounts (% by weight) |
|---|---|
| FGF6 (from transgenic plant) | 0.0005 |
| Propyleneglycol | 2.0 |
| Glycerin | 4.0 |
| Carboxyvinylpolymer | 0.3 |
| Ethanol | 7.0 |
| PEG-40 Hydrogenated Castor Oil | 0.8 |
| Triethanolamine | 0.3 |
| Flavor | 0.2 |
| Methylparaben | 0.2 |
| Imidazolidinyl Urea | 0.2 |
| Purified water | q.s. |

The formulations 1-5 can likewise be formulated with any alternative growth factor selected from those listed in the Detailed description.

### Formulation 6: Solution for Parenteral Use

58 mg of lyophilized substance, comprising 25 ug of active substance and 33 mg of phosphate buffer (10 mg NaH₂PO₄/H₂O and 23 mg Na₂HPO₄/2H₂O), and about 125 ml physiological solution for parenteral use, are separately packaged in flasks preset for mixing the lyophilized product with the diluent immediately prior to use. The post-solubilisation concentration of active substance is of about 0.2 ug/ml. The active substance can suitably be selected from any of the growth factors listed in the description, which growth factor is trangenically expressed in a plant and isolated therefrom, as described herein.

### Formulation 7: W/O Emulsion for Topical Application.

An amount of lyophilized substance comprising 20 ug active substance is brought to 5 ml 10% ethanol hydro-alcoholic solution comprising 10% DMSO. The solution is emulsified in sterilised vegetable oil for cutaneous application using a surfactant suitable for W/O emulsions having a <10 HLB coefficient. The emulsion contains active substance equal to about 2 ug/g of composition. The active substance is a plant-derived recombinant growth factor selected from the listed growth factors in the description and isolated from the host plant.

### Formulation 8: O/W Emulsion

An amount of lyophilized substance comprising about 20 ug active substance is solubilised in 5 ml of hydro-alcoholic solution comprising 30% DMSO and emulsified with a suitable surfactant in a vegetable oil-based lipophilic solvent. The resulting O/W emulsion contains the active substance at a concentration of about 3 ug/g composition. The active substance is a plant-derived recombinant growth factor selected from the listed growth factors in the description and isolated from the host plant.

### Formulation 9: Topical Composition in Form of Gel.

An amount of lyophilized substance comprising 10 ug of active substance is brought in 20 ml 10% ethanol hydro-alcoholic solution. Then, the solution is additioned with a mixture of pentylene glycol, aminomethyl propanol and acrylates, caprylyl glycol and tropolone. The active substance is present in an amount equal to 0.2 ug/g composition. The gel is suitable for cosmetic application. The active substance is a plant-derived recombinant growth factor selected from the listed growth factors in the description and isolated from the host plant.

### Formulation 10: A Topical Gel Formulation Containing Carbomer (1%)

| Ingredients | Amounts (% by weight) | |
|---|---|---|
| Hb-EGF (from transgenic plant) | 5 | ug |
| Carbomer 934P | 1 | g |
| Methyl paraoxybenzoate | 0.2 | g |
| Propylene glycol | 20 | g |
| Sodium hydroxide | q.s | |
| Distilled water for injection | q.s | |
| Total | 100 | g |

The formulation is prepared by using the above-mentioned components in given amounts according to a conventional method. Specifically, methyl paraoxybenzoate is dissolved in appropriate amounts of distilled water for injection, Carbomer 934P is added to the solution and dispersed therein with sting. The pH of the solution is controlled with sodium hydroxide, the solution is blended with propylene glycol and sterilized by heating. Then, filtered and sterilized solution of Hb-EGF in distilled water for injection is added thereto to obtain 100 g of formulation.

### Formulation 10: A Topical Formulation Containing Poloxamer(20%)

| Ingredients | Amounts (% by weight) | |
|---|---|---|
| Hb-EGF | 5 | ug |
| Poloxamer 407 | 20 | g |
| Methyl paraoxybenzoate | 0.2 | g |
| Sodium hydrogen phosphate | 272.18 | mg |
| Sodium chloride | 666.22 | mg |
| Phosphoric acid | q.s | |
| Propylene glycol | 20 | g |
| Distilled water for injection | q.s. | |
| Total | 100 | g |

The formulation is prepared by using the above-mentioned components in given amounts according to a conventional method. Specifically, phosphate buffer is prepared by using sodium hydrogen phosphate, sodium chloride and phosphoric acid in given amounts. Methyl paraoxybenzoate as the preservative is dissolved to the phosphate buffer. Poloxamer 407(BASF, Germany) is added to the solution and dispersed therein with string. Then the solution is blended with propylene glycol, dispersed therein with stirring. Then, the pH of the solution is controlled with sodium hydroxide, the solution is blended with propylene glycol and sterilized by heating. Then, filtered and sterilized solution of Hb-EGF in distilled water for injection is added thereto to obtain 100 g of formulation.

### Formulation 11: A Cream Formulation Containing Carbomer (0.1%)

| Ingredients | Amounts (% by weight) | |
|---|---|---|
| Hb-EGF | 0.05 | mg |
| Glycerin | 4.5 | g |
| Methyl paraoxybenzoate | 0.15 | g |
| Propyl paraoxybenzoate | 0.05 | g |
| Carbomer 940 | 0.1 | g |
| Steary alcohol | 1.75 | g |
| Cetyl alcohol | 4.00 | g |
| Span #60 | 0.50 | g |
| Polyoxyl #40 stearate | 2.00 | g |
| Triethanolamine | q.s | |
| Distilled water for injection | q.s | |
| Total | 100 | g |

The formulation is prepared by using the above-mentioned components in given amounts according to a conventional method. Specifically, glycerin and methyl paraoxybenzoate are dissolved in appropriate amounts of distilled water for injection, Carbomer 940(BF Goodrich, U.S.A.) is added to the solution and dispersed therein with stirring. Then, propyl paraoxybenzoate and the others are added to the solution and emulsified with melting. Then, the solution is sterilized after controlling pH with triethanolamine, and mixed with filtered and sterilized solution of recombinant Hb-EGF expressed and isolated from plant) in distilled water for injection to obtain 100 g of formulation.

1. A cosmetic and/or therapeutic composition comprising a recombinant non-plant, heterologous growth factor derived from a transgenic plant.

2. The composition of claim 1, which is a topical composition for application to skin.

3. The composition of claim 1, wherein the growth factor is provided as a component of a transgenic plant extract comprised in the cosmetic composition.

4. The composition of claim 3, where the growth factor is present in the transgenic plant extract in amount in the range of about 0.01% to about 70% of the total protein content.

5. The composition of claim 3, where the growth factor is present in the transgenic plant extract in an amount, more specifically, in the range of about 0.1% to about 30% of the total protein content.

6. The composition of claim 1, comprising more than one growth factor derived from transgenic plants.

7. The composition of claim 6, where said more than one growth factor are present as components of a mixture of extracts from transgenic plants.

8. A cosmetic and/or therapeutic composition comprising a plant extract from a transgenic plant expressing a recombinant heterologous non-plant growth factor.

9. The composition of claim 1, which is a topical composition for application to skin.

10. The composition of claim 8, wherein said plant extract comprises in the range of about 0.01% to about 99.9% of said growth factor, and preferably in the range of about 0.1 to about 99.9%.

11. The composition of claim 8, wherein said plant extract comprises in the range of about 0.01% to about 70% of said growth factor, such as in the range of about 0.1% to about 40%, or in the range of about 40% to about 70%.

12. The composition of claim 8, wherein one or more plant-derived purified non-plant growth factors are added to said plant extract already containing a non-plant growth factor.

13. The composition of claim 1, wherein said growth factor is glycosylated with plant-specific glycosylation.

14. The composition of claim 1, where the growth factor is selected from the group consisting of Epidermal Growth Factor (EGF), Vascular Epithelial Growth Factor (VEGF), Platelet-Derived Growth Factor (PDGF), Erythropoietin (Epo), Fibroblast growth factors 4, 5, 6, 8 and 9 (FGF4, FGF5, FGF6, FGF8 and FGF9), Fibroblast Growth Factors a and b, Flt3 ligand, Heparin binding-EGF (Hb-EGF), Insulin-Like Growth Factor-I (IGF-I), Insulin-Like Growth Factor-II (IGF-II), Interleukin-1 (IL-1; including IL-1 alpha and IL 1-beta), Interleukin-2 (IL-2), Interleukin-4 (IL-4), Interleukin-7 (IL-7), Interleukin-6 (IL-6), Interleukin-8 (IL-8), Interleukin-10 (IL-10), Interleukin-15 (IL-15), Interleukin-18 (IL-18), Interleukin-20 (IL-20), leukemia inhibitory factor (LIF), Noggin, Placenta growth factor-1 (PlGF-1), Stem cell factor (SCF), Transforming growth factor alfa and beta (TGF a and TGF b), including TGF b3 Tumor Necrosis Factor-a (TNF-a), Tumor Necrosis Factor-b (TNF-b), Interferon-g (INF-g) Granulocyte Colony Stimulating Factor (G-CSFs), Granulocyte Macrophage Colony Stimulating Factor (GM-CSF), Macrophage Colony stimulating factor (M-CSF), Nerve Growth Factor (NGF), Keratinocyte Growth Factor (KGF), Bone morphogenesis Protein (BMP-4), and Thymosin beta 4.

15. The composition of claim 3, wherein said plant extract comprises a protein originating from the extracted plant, selected from the group consisting of dehydrins, globulins and other seed proteins.

16. The cosmetic composition of claim 1, wherein the composition is in the form selected from the group consisting of creams, lotions, gels, dressings, shampoos, tinctures, pastes, ointments, salves, powders, liquid or semiliquid formulations, patches, liposomal preparations, solutions, suspensions, liposome suspensions, W/O or O/W emulsions, ointments, pomades and pastes and a skin softener cream, a facial pack, a massage cream, and a nutrient cream or a nutrient emulsion.

17. A method of manufacturing a topical skin care product comprising
providing a plant extract from a transgenic plant expressing a heterologous non-plant growth factor, and combining with at least one cosmetically acceptable excipient.

18. The method of claim 17, wherein said method further comprises harvesting a transgenic plant expressing said heterologous growth factor, extracting from the plant material said plant extract.

19. The method of claim 17, wherein said plant extract comprises in the range of about 0.01% wt to about 70 wt % of said heterologous growth factor.

20. The method of claim 17, wherein said plant extract comprises a protein originating from the extracted plant, selected from the group consisting of dehydrins, globulins and other seed proteins.

21. The method of claim 17, wherein said transgenic plant extract is barley seed extract.

22. The method of claim 17, wherein said heterologous growth factor is selected from the group consisting of Transforming Growth Factors-b (or beta) (TGFs-b or TGFs-beta), Transforming Growth Factor-a (or alpha) (TGF-a or TGF alpha), TNF alpha, Epidermal Growth Factor (EGF), BMP-4, Platelet-Derived Growth Factor (PDGF), KGF, Fibroblast Growth Factors a and (aFGF and bFGF), Vascular Epithelial Growth Factor (VEGF) Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-7 (IL-7), Interleukin-6 (IL-6), Interleukin-8 (IL-8), Interleukin-10 (IL-10), Interleukin-18 (IL-18), Interleukin-20 (IL-20), Tumor Necrosis Factor-a (TNF-a), Tumor Necrosis Factor-b (TNF-b), Interferon-g (INF-g), Granulocyte Colony Stimulating Factor (G-CSFs), Granulocyte Macrophage Colony Stimulating Factor (GM-CSF), Macrophage Colony stimulating factor (M-CSF), Nerve Growth Factor (NGF), Keratinocyte Growth Factor (KGF), Bone morphogenesis Protein (BMP-4), and Thymosin beta 4.

23. The method of claim 17, further comprising isolating said heterologous growth factor from said transgenic plant extract.

24. The method of claim 23, wherein said step of isolating comprises using affinity chromatography.

25. The method of claim 23, where said step of isolating comprises using ion exchange chromatography.
